(19) United States
(12) Patent Application Publication
(10) Pub. No.: US 2018/0110802 A1
(43) Pub. Date: Apr. 26, 2018

(54) MINERAL WATER COMPOSITION

(71) Applicant: MINERAL ELEMENT GROUP CORPORATION, Houston, TX (US)

(72) Inventors: Jianyi Xi, Qingdao (CN); Guangcheng Liu, Qingdao (CN)

(73) Assignee: MINERAL ELEMENT GROUP CORPORATION, Houston, TX (US)

(21) Appl. No.: 15/792,184

(22) Filed: Oct. 24, 2017

Related U.S. Application Data
(60) Provisional application No. 62/412,479, filed on Oct. 25, 2016.

(51) Int. Cl. A61K 33/00; A61K 9/00; A61K 33/38; A61K 33/44; A61K 33/42; A61K 33/06; A61K 33/34; A61K 33/32; A61K 33/30; A61K 33/26; A61K 33/24; A61K 33/22; A61K 33/04; A23L 33/16; A23L 33/00

(52) U.S. Cl. CPC A61K 33/00; A23V 2002/00; A61K 9/0095; A61K 33/38; A61K 33/44; A61K 33/42; A61K 33/06; A61K 33/34; A61K 33/32; A61K 33/30; A61K 33/26; A61K 33/24; A61K 33/22; A61K 33/245; A61K 33/04; A23L 33/16; A23L 33/40; A61K 9/0014

(57) ABSTRACT

A mineral water comprising a composition of a blend of minerals and trace rare earth elements is provided, the rare earth elements in the composition comprise Scandium, Yttrium, Lanthanum, Cerium, Praseodymium, Neodymium, Samarium, Europium, Gadolinium, Terbium, Dysprosium, Holmium, Erbium, Thulium, Ytterbium, Lutetium. The water is conducive to good health and can relieve the symptoms of some chronic diseases.

### CROSS-REFERENCE TO RELATED APPLICATIONS

This application claims the benefit of U.S. Provisional Patent Application No. 62/412,479, filed Oct. 25, 2016; incorporated herein by reference in its entirety.

### FIELD

The disclosure relates generally to a consumable mineral water. The disclosure relates specifically to a mineral water composition containing a blend of minerals and trace rare earth elements.

### BACKGROUND

The need for minerals and trace elements are well known in the nutritional and holistic arts. Potassium, sodium, calcium, and magnesium ions are the predominant metal ions in the cytoplasm of a cell. Many of these minerals and trace elements are used as cofactors, activators, or stabilizers bound to enzymes, proteins, nucleic acids or other biochemicals. Calcium ion combines with phosphate ion to comprise bone matrix. Iron is at the center of a heme group that make up hemoglobin, a metalloprotein that binds oxygen for respiration. Magnesium or manganese ion binds with adenosine triphosphate (ATP) to form the absolutely required substrate used in most ATP-dependent enzymatic reactions. The trace element selenium is required by the enzyme superoxide dismutase for effective catalysis. Fluoride binds to the enamel of your teeth, thereby inhibiting tooth decay. Iodine is another trace element needed for proper function of the thyroid gland. Silicon is an essential element required for normal growth development, especially with bones and connective tissue.

The use of rare earth elements provides certain health benefits. The applications of rare earths described by scientific literature include treatment of burns (M. Deveci, et al., Effects of cerium nitrate bathing and prompt burn wound excision on IL-6 and TNF-α levels in burned rats. Burns, 26(1):41-45, 2000), diagnosis and treatment of cancer (Y. Dai, et al., Effects of rare earth compounds on growth and apoptosis of leukemic cell lines. In Vitro Cellular and Development Biology-Animal, 38:373-375, 2002), treatment of joint diseases (Ofluoglu, et al., Radiation synovectomy with (166) Ho-ferric hydroxide: a first experience. Journal of Nuclear Medicine, 43(11):1489-1494, 2002), prevention of caries (K. Collys, et al. A comparison of the influence of lanthanum and fluoride on de- and remineralization of bovine enamel in vitro. Journal of Dental Research, 69(2):458-462, 1990), prevention of atherosclerosis (W. Zhu, et al., Investigation on arteriosclerosis among population in a rare earth area in south China. Biological Trace Element Research, 59(1-3):93-98, 1997), treatment of osteoporosis (J. Beherts, et al., Localization of lanthanum in bone of chronic renal failure rats after oral dosing with lanthanum carbonate. Kidney International, 67(5):1830, 2005). Further possible health applications have been suggested including antidiabetic actions, therapy of skin diseases, prevention of immune-related rejection following organ transplantation as well as inhibition of scar formation (H. Evans. Biochemistry of the lanthanides. Plenum Press, New York and London, 1990)

### SUMMARY

The present invention is directed to a mineral water comprising a health promoting composition of a blend of minerals and trace rare earth elements. The rare earth elements in the composition comprise Scandium(Sc), Yttrium(Y), Lanthanum(La), Cerium(Ce), Praseodymium(Pr), Neodymium(Nd), Samarium(Sm), Europium(Eu), Gadolinium(Gd), Terbium(Tb), Dysprosium(Dy), Holmium(Ho), Erbium(Er), Thulium(Tm), Ytterbium(Yb), Lutetium(Lu). The concentration range of the rare earth elements are: from about 80 to 120 micrograms of Scandium, from about 8 to 20 micrograms of Yttrium, from about 0.1 to 0.15 micrograms of Lanthanum, from about 0.2 to 0.3 micrograms of Cerium, from about 0.02 to 0.05 micrograms of Praseodymium, from about 0.12 to 0.15 micrograms of Neodymium, from about 0.02 to 0.04 micrograms of Samarium, from about 0.002-0.01 micrograms of Europium, from about 0.02-0.04 micrograms of Gadolinium, from about 0.002-0.01 micrograms of Terbium, from about 0.01-0.03 micrograms of Dysprosium, from about 0.002 to 0.01 micrograms of Holmium, from about 0.01 to 0.03 micrograms of Erbium, from about 0.0002 to 0.0008 micrograms of Thulium, from about 0.01 to 0.03 micrograms of Ytterbium and from about 0.0002 to 0.0008 micrograms of Lutetium.

The chemical elements in a preferred embodiment, the composition further include: Lithium, Beryllium, Boron, Carbon, Nitrogen, Sodium, Magnesium, Aluminum, Silicon, Phosphorous, Potassium, Calcium, Titanium, Vanadium, Chromium, Manganese, Iron, Cobalt, Nickel, Copper, Zinc, Gallium, Germanium, Selenium, Rubidium, Strontium, Zirconium, Niobium, Molybdenum, Ruthenium, Rhodium, Palladium, Silver, Indium, Tin, Antimony, Tellurium, Cesium, Barium, Hafnium, Tantalum, Tungsten, Rhenium, Osmium, Iridium, Platinum, Gold, Thallium, Bismuth, Thorium. The concentration range of these elements are: from about 5 to 10 micrograms of Lithium, from about 0.05 to 0.15 micrograms of Beryllium, from 0.5 to 1.0 micrograms of Boron, from about 100 to 150 micrograms of Carbon, 10.5 micrograms of Nitrogen, from about 100 to 150 micrograms of Sodium, from 1000 to 8000 micrograms of Magnesium, from 1000 to 5000 micrograms of Aluminum, from 10 to 20 micrograms of Silicon, from 15 to 25 micrograms of Phosphorous, from 100 to 400 micrograms of Potassium, from 0 to 10 micrograms of Calcium, from 50 to 120 micrograms of Titanium, from 1 to 4 micrograms of Vanadium, from 20 to 40 micrograms of Chromium, from 20 to 40 micrograms of Manganese, from 1500 to 2500 micrograms of Iron, from 1 to 2.5 micrograms of Cobalt, from 15 to 30 micrograms of Nickel, from 1 to 3 micrograms of Copper, from 2 to 5 micrograms of Zinc, from 0.2 to 0.8 micrograms of Gallium, from 0.002 to 0.01 micrograms of Germanium, from 0.02 to 0.1 micrograms of Selenium, from 1.2 to 3 micrograms of Rubidium, from 1 to 3 micrograms of Strontium, from 0.1 to 0.2 micrograms of Zirconium, from 0.01 to 0.04 micrograms of Niobium, from 0.02 to 0.06 micrograms of Molybdenum, from 0.0001 to 0.0008 micrograms of Ruthenium, from 0.0001 to 0.0008 micrograms of Rhodium, from 0.002 to 0.01 micrograms of Palladium, from 0.0001 to 0.0008 micrograms of Silver, from 0.0001 to 0.0008 micrograms of Indium, from 0.01 to 0.05 micrograms of Tin, from 0.0001 to 0.0008 micrograms of Antimony, from 0.0001 to 0.0008 micrograms of Tellurium, from 0.05 to 0.25 micrograms of Cesium, from 0.01 to 0.05 micrograms of Barium, from 0.0001 to 0.0008 micrograms of Hafnium, from 0.002 to 0.01 micrograms of Tantalum, from 0.001 to 0.008 micrograms of Tungsten, from 0.0001 to 0.0008 micrograms of Rhenium, from 0.0001 to 0.0008 micrograms of Osmium, from 0.0001 to 0.0008 micrograms of Iridium, from 0.0001 to 0.0008 micrograms of Platinum, from 0.001 to 0.01 micrograms of Gold, from 0.01 to 0.07 micrograms of Thallium, from 0.0001 to 0.0008 micrograms of Bismuth, from 0.2 to 1 micrograms of Thorium.

The mineral water is effective to agitate and increase the efficiency of human body enzyme activity, maintain and balance body's nutrition, dissolve excess body fat, cleanse blood, increase the metabolism of the body, sharply increase the immunity of the body and improve body's self-healing capability. The mineral water is also capable to enable body cells hold adequate water content, prevent the skin from getting rough, and it can increase body energy and prevent the aging of the body.

The foregoing has outlined rather broadly the features of the present disclosure in order that the detailed description that follows may be better understood. Additional features and advantages of the disclosure will be described hereinafter, which form the subject of the claims.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

Preparation of the Mineral Water

The minerals and trace rare earth elements are taken from granite using modern scientific techniques and biology techniques.

The granite used in the present disclosure is chinese maifan stone which is produced in Pingdingshan, inner Mongolia of china. The main chemical component of the Chinese maifan stone is aluminosilicate, it includes Si₂O₃, Fe₂O₃, FeO, MgO, CaO, k₂O, Na₂O, TiO₂, p₂O₅0, MnO, and contains all the major elements required for animals, such as: K, Na, Ca, Mg, Cu, Mo and other trace elements and rare earth elements. After soaked in water, potassium, sodium, calcium, magnesium, silicon, manganese, titanium, phosphorus and other essential elements can be dissolved from the maifan stone. The water can reach mineral water standards, long-term drinking can enhance the body's immune function, improve the body's resistance to infection. But many beneficial elements are not dissolved in the water. In order to dissolve these elements, the chinese maifan stone is crushed to about 1.5 microns powder, and the powder is put into H₂SO₄ solution, heating, pressuring and stirring the solution, when the components are dissolved in the solution, calcium hydroxide is put into the solution to neutralize some SO₄ ion in the solution. After that, the solution is filtered for the first time to get rid of residue and then is treated to adjust the concentration. Following the concentration, The solution is settled to make impurities to be settled, following the settling, the solution is filtered for the second time and at last the mineral water is produced. The components beneficial to human are dissolved in the mineral water in ionic form. The pH value of the mineral water is 0.85. To use the mineral water, the mineral water is diluted 500-1000 times with water, such that the pH value is above 6 which can be directly drunk by human being.

Table 1 shows the elements of a sample from a preferred embodiment. The sample was tested by Advanced Botanical Consulting & Testing Inc.

TABLE 1

| Element | [micrograms] | element | [micrograms] |
|---|---|---|---|
| Lithium (Li) | 8.99 | Palladium (Pd) | 0.005 |
| Beryllium (Be) | 0.105 | Silver (Ag) | <0.001 |
| Boron (B) | 0.84 | Indium (In) | <0.001 |
| Carbon (C) | 129.34 | Tin (Sn) | 0.035 |
| Nitrogen (N) | 10.5 | Antimony (Sb) | <0.001 |
| Sodium (Na) | 124.495 | Tellurium (Te) | <0.001 |
| Magnesium (Mg) | 7637.78 | Cesium (Cs) | 0.12 |
| Aluminum (Al) | 3131.345 | Barium (Ba) | 0.03 |
| Silicon (Si) | 15.535 | Lanthanum (la) | 0.12 |
| Phosphorous (P) | 22.105 | Cerium (Ce) | 0.275 |
| Potassium (K) | 351 | Praseodymium (Pr) | 0.035 |
| Calcium (Ca) | 0 | Neodymium (Nd) | 0.14 |
| Scandium (Sc) | 0.405 | Samarium (Sm) | 0.03 |
| Titanium (Ti) | 118.945 | Europium (Eu) | 0.005 |
| Vanadium (V) | 3.05 | Gadolinium (Gd) | 0.035 |
| Chromium (Cr) | 28.56 | Terbium (Tb) | 0.005 |
| Manganese (Mn) | 31.38 | Dysprosium (Dy) | 0.025 |
| Iron (Fe) | 1932.865 | Holmium (Ho) | 0.005 |
| Cobalt (Co) | 1.895 | Erbium (Er) | 0.015 |
| Nickel (Ni) | 22.235 | Thulium (Tm) | <0.001 |
| Copper (Cu) | 1.89 | Ytterbium (Yb) | 0.015 |
| Zinc (Zn) | 4.205 | Lutetium (Lu) | <0.001 |
| Gallium (Ga) | 0.445 | Hafnium (Hf) | <0.001 |
| Germanium (Ge) | 0.005 | Tantalum (Ta) | <0.001 |
| Selenium (Se) | 0.055 | Tungsten (W) | 0.005 |
| Rubidium (Rb) | 2.325 | Rhenium (Re) | <0.001 |
| Strontium (Sr) | 1.225 | Osmium (Os) | <0.001 |
| Yttrium (Y) | 12.415 | Iridium (Ir) | <0.001 |
| Zirconium (Zr) | 0.115 | Platinum (Pt) | <0.001 |
| Niobium (Nb) | 0.025 | Gold (Au) | 0.005 |
| Molybdenum (Mo) | 0.045 | Thallium (Tl) | 0.025 |
| Ruthenium (Ru) | <0.001 | Bismuth (Bi) | <0.001 |
| Rhodium (Rh) | <0.001 | Thorium (Th) | 0.565 |

The anions in the sample are shown in Table 2

TABLE 2

| Chemical Component | Concentration, % w/w |
|---|---|
| SO4²⁻ | 7.566 |
| PO4³⁻ | 0 |
| Cl⁻ | 0.0046 |
| NO₃ | 0.0642 |

The loss on drying of the mineral water is 92.2329% and the mineral water has a pH of 0.85.

Results

The health care and healing capabilities of the mineral water include but are not limited to:

In an embodiment for skin rashes, skin infection and allergy: apply the mineral water to the infected area using gauze, the skin disease will be healed about ten days after using the mineral water. In an embodiment, various days of treatment can be used.

In an embodiment for psoriasis treatment, soak in 1% mineral water bath besides apply it to the infected area. In an embodiment, varying concentrations can be used.

In an embodiment for gout: dissolve 10 ml of the mineral water using pure water first and then wash the affected area two times a day, typically, the affected area will recover significantly for one month. In an embodiment, various amounts can be used multiple times a day for various numbers of days.

In an embodiment for gastric ulcers: Drink 10 ml of the mineral water one time every day 30 minutes before going to bed; the symptoms will be significantly improved after 10 days of use, and it will be fully cured after one-month of use. In an embodiment, various amounts can be for various numbers of days.

In an embodiment for hypertension and nephritis: dissolve 10 ml of the mineral water using pure water first and then drink 10 ml the mixed water, two times a day, one in the morning and one at night, typically, the symptom will be significantly improved for three months of use. In an embodiment, various amounts can be used multiple times a day for various numbers of days.

In an embodiment for diabetes (Type II): dissolve 10 ml of the mineral water using pure water first and then drink 10 ml of the mixed water, two times a day, one in the morning and one at night, typically, the symptom will be significantly improved for six months of use. In an embodiment, various amounts can be used multiple times a day for various numbers of days.

In an embodiment for healthy individuals: Take 10 ml the mineral water every day will make people feel energized and experience longevity. People who take the mineral water throughout the year will keep the body system in a weak alkaline state. In an embodiment, various amounts can be used for various numbers of days.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A mineral water, comprising a composition of a blend of minerals and trace rare earth elements comprising Scandium(Sc), Yttrium(Y), Lanthanum(La), Cerium(Ce), Praseodymium(Pr), Neodymium(Nd), Samarium(Sm), Europium(Eu), Gadolinium(Gd), Terbium(Tb), Dysprosium(Dy), Holmium(Ho), Erbium(Er), Thulium(Tm), Ytterbium(Yb), and Lutetium(Lu).

2. The mineral water of claim 1 wherein the concentration range of the rare earth elements comprises from about 0.1 to 1 micrograms of Scandium, from about 8 to 20 micrograms of Yttrium, from about 0.1 to 0.15 micrograms of Lanthanum, from about 0.2 to 0.3 micrograms of Cerium, from about 0.02 to 0.05 micrograms of Praseodymium, from about 0.12 to 0.15 micrograms of Neodymium, from about 0.02 to 0.04 micrograms of Samarium, from about 0.002-0.01 micrograms of Europium, from about 0.02-0.04 micrograms of Gadolinium, from about 0.002-0.01 micrograms of Terbium, from about 0.01-0.03 micrograms of Dysprosium, from about 0.002 to 0.01 micrograms of Holmium, from about 0.01 to 0.03 micrograms of Erbium, from about 0.0002 to 0.0008 micrograms of Thulium, from about 0.01 to 0.03 micrograms of Ytterbium, and from about 0.0002 to 0.0008 micrograms of Lutetium.

3. The mineral water of claim 2, wherein the concentration of the rare earth are: 0.405 micrograms of Scandium, 12.415 micrograms of Yttrium, 0.12 micrograms of Lanthanum, 0.275 micrograms of Cerium, from about 0.035 micrograms of Praseodymium, 0.14 micrograms of Neodymium, 0.03 micrograms of Samarium, 0.005 of Europium, 0.035 micrograms of Gadolinium, 0.005 micrograms of Terbium, 0.025 of Dysprosium, 0.005 micrograms of Holmium, 0.015 micrograms of Erbium, 0.0008 micrograms of Thulium, 0.015 micrograms of Ytterbium and 0.0008 micrograms of Lutetium, the concentration of the mineral elements are: 8.99 micrograms of Lithium, 0.105 micrograms of Beryllium, 0.84 micrograms of Boron, 129.34 micrograms of Carbon, 10.5 micrograms of Nitrogen, 124.495 micrograms of Sodium, 7637.78 micrograms of Magnesium, 3131.345 micrograms of Aluminum, 15.535 micrograms of Silicon, 22.105 micrograms of Phosphorous, 351 micrograms of Potassium, 0 micrograms of Calcium, 118.945 micrograms of Titanium, 3.05 micrograms of Vanadium, 28.56 micrograms of Chromium, 31.38 micrograms of Manganese, 1932.865 micrograms of Iron, 1.895 micrograms of Cobalt, 22.235 micrograms of Nickel, 1.89 micrograms of Copper, 4.205 micrograms of Zinc, 0.445 micrograms of Gallium, 0.005 micrograms of Germanium, 0.055 micrograms of Selenium, 2.325 micrograms of Rubidium, 1.225 micrograms of Strontium, 0.115 micrograms of Zirconium, 0.025 micrograms of Niobium, 0.045 micrograms of Molybdenum, 0.0008 micrograms of Ruthenium, 0.0008 micrograms of Rhodium, 0.005 micrograms of Palladium, 0.0008 micrograms of Silver, 0.0008 micrograms of Indium, 0.035 micrograms of Tin, 0.0008 micrograms of Antimony, 0.0008 micrograms of Tellurium, 0.12 micrograms of Cesium, 0.03 micrograms of Barium, 0.0008 micrograms of Hafnium, 0.005 micrograms of Tantalum, 0.008 micrograms of Tungsten, 0.0008 micrograms of Rhenium, 0.0008 micrograms of Osmium, 0.0008 micrograms of Iridium, 0.0008 micrograms of Platinum, 0.005 micrograms of Gold, 0.025 micrograms of Thallium, 0.0008 micrograms of Bismuth, and 0.565 micrograms of Thorium.

4. The mineral water of claim 1, wherein the minerals comprise Lithium, Beryllium, Boron, Carbon, Nitrogen, Sodium, Magnesium, Aluminum, Silicon, Phosphorous, Potassium, Calcium, Titanium, Vanadium, Chromium, Manganese, Iron, Cobalt, Nickel, Copper, Zinc, Gallium, Germanium, Selenium, Rubidium, Strontium, Zirconium, Niobium, Molybdenum, Ruthenium, Rhodium, Palladium, Silver, Indium, Tin, Antimony, Tellurium, Cesium, Barium, Hafnium, Tantalum, Tungsten, Rhenium, Osmium, Iridium, Platinum, Gold, Thallium, Bismuth, and Thorium.

5. The mineral water of claim 4, wherein the concentration range of these elements comprises from about 5 to 10 micrograms of Lithium, from about 0.05 to 0.15 micrograms of Beryllium, from 0.5 to 1.0 micrograms of Boron, from about 100 to 150 micrograms of Carbon, 10.5 micrograms of Nitrogen, from about 100 to 150 micrograms of Sodium, from 1000 to 8000 micrograms of Magnesium, from 1000 to 5000 micrograms of Aluminum, from 10 to 20 micrograms of Silicon, from 15 to 25 micrograms of Phosphorous, from 100 to 400 micrograms of Potassium, from 0 to 10 micrograms of Calcium, from 50 to 120 micrograms of Titanium, from 1 to 4 micrograms of Vanadium, from 20 to 40 micrograms of Chromium, from 20 to 40 micrograms of Manganese, from 1500 to 2500 micrograms of Iron, from 1 to 2.5 micrograms of Cobalt, from 15 to 30 micrograms of Nickel, from 1 to 3 micrograms of Copper, from 2 to 5 micrograms of Zinc, from 0.2 to 0.8 micrograms of Gallium, from 0.002 to 0.01 micrograms of Germanium, from 0.02 to 0.1 micrograms of Selenium, from 1.2 to 3 micrograms of Rubidium, from 1 to 3 micrograms of Strontium, from 0.1 to 0.2 micrograms of Zirconium, from 0.01 to 0.04 micrograms of Niobium, from 0.02 to 0.06 micrograms of Molybdenum, from 0.0001 to 0.0008 micrograms of Ruthenium, from 0.0001 to 0.0008 micrograms of Rhodium, from 0.002 to 0.01 micrograms of Palladium, from 0.0001 to 0.0008 micrograms of Silver, from 0.0001 to 0.0008 micrograms of Indium, from 0.01 to 0.05 micrograms of Tin, from 0.0001 to 0.0008 micrograms of Antimony, from 0.0001 to 0.0008 micrograms of Tellurium, from 0.05 to 0.25 micrograms of Cesium, from 0.01 to 0.05 micrograms of Barium, from 0.0001 to 0.0008 micrograms of Hafnium, from 0.002 to 0.01 micrograms of Tantalum, from 0.001 to 0.008 micrograms of Tungsten, from 0.0001 to 0.0008 micrograms of Rhenium, from 0.0001 to 0.0008 micrograms of Osmium, from 0.0001 to 0.0008 micrograms of Iridium, from 0.0001 to 0.0008 micrograms of Platinum, from 0.001 to 0.01 micrograms of Gold, from 0.01 to 0.07 micrograms of Thallium, from 0.0001 to 0.0008 micrograms of Bismuth, from 0.2 to 1 micrograms of and Thorium.
